(19) United States
(12) Patent Application Publication
(10) Pub. No.: US 2014/0316286 A1
(43) Pub. Date: Oct. 23, 2014

(54) SYSTEM AND METHOD FOR DETERMINING RESPIRATORY PARAMETERS FROM BLOOD FLOW SIGNALS

(71) Applicant: COVIDIEN LP, Boulder, CO (US)

(72) Inventors: Paul Stanley Addison, Edinburgh (GB); James Nicholas Watson, Dunfermline (GB)

(73) Assignee: Covidien LP, Boulder, CO (US)

(21) Appl. No.: 13/867,291

(22) Filed: Apr. 22, 2013

(51) Int. Cl. A61B 5/00; A61B 8/06; A61B 6/03; A61B 6/00; A61B 5/026; A61B 5/0205; A61B 8/08

(52) U.S. Cl. CPC A61B 5/7278; A61B 5/0205; A61B 5/726; A61B 8/06; A61B 8/488; A61B 6/504; A61B 5/0261; A61B 5/0263; A61B 6/037; A61B 5/0816
USPC 600/484

(57) ABSTRACT

A system for determining one or more respiratory parameters of an individual may include a blood flow detection device configured to detect a blood flow signal of the individual, a blood flow determination module configured to form a blood flow waveform based on the blood flow signal, and a respiratory parameter analysis module configured to analyze the blood flow waveform and determine the respiratory parameter(s) from an analysis of the blood flow waveform.

### FIELD

Embodiments of the present disclosure generally relate to physiological signal processing and, more particularly, to a system and method that determines one or more respiratory parameters, such as respiration rate and respiratory effort, from blood flow signals.

### BACKGROUND

Respiration rate, also known as respiratory rate, pulmonary ventilation rate, ventilation rate, or breathing frequency, generally represents the number of breaths taken within a predetermined time frame, such as within a sixty second time frame. In general, respiration rate may be measured when an individual is at rest, and involves counting the number of breaths, such as by counting how many times the chest of the individual rises.

However, detection of respiration rate may be hindered through mechanical or other physical connections to a patient. Moreover, many systems currently designed to detect respiration rate may not always be simple and easy to use. Further, certain detection systems may not be quickly and easily used with other patient monitoring systems.

### SUMMARY

Certain embodiments of the present disclosure provide a system for determining one or more respiratory parameters of an individual. The system may include a blood flow detection device configured to detect a blood flow signal of the individual, a blood flow determination module configured to form a blood flow waveform based on the blood flow signal, and a respiratory parameter analysis module configured to analyze the blood flow waveform and determine the respiratory parameter(s) from an analysis of the blood flow waveform.

The respiratory parameter(s) may include respiration rate. The respiratory parameter analysis module may be configured to determine the respiration rate by correlating a periodicity of the blood flow waveform with breaths of the individual, or vice versa.

The respiratory parameter(s) may include respiratory effort. The respiratory parameter analysis module may be configured to determine the respiratory effort through a determination of one or both of an amplitude or frequency of the blood flow waveform. The respiratory effort may be directly proportional to one or both of the amplitude or the frequency of the blood flow waveform.

The respiratory parameter analysis module may be configured to determine the respiratory parameter(s) through a correlation of portions of the blood flow waveform with breathing characteristics of the individual. Alternatively, the respiratory parameter analysis module may include a wavelet transform module configured to transform the blood flow waveform into a scalogram using at least one wavelet transform, and a wavelet analysis module configured to determine the one or more respiratory parameters through an analysis of the scalogram.

The blood flow detection device may be configured to detect the blood flow signal through one or more of Doppler ultrasound, angiography, laser Doppler flowmetry, optical Doppler tomography, magnetic resonance angiography, microscopic video imaging, laser speckle imaging, optical coherent tomography, positron emission tomography, confocal microscopy, ultrasound imaging, or orthogonal polarization spectral imaging.

Certain embodiments of the present disclosure provide a method for determining one or more respiratory parameters of an individual. The method may include detecting a blood flow signal of an individual with a blood flow detection device, forming a blood flow waveform based on the blood flow signal with a blood flow determination module, analyzing the blood flow waveform with a respiratory parameter analysis module, and determining, with the respiratory analysis module, the respiratory parameter(s) through the analyzing operation.

Certain embodiments of the present disclosure provide a tangible and non-transitory computer readable medium that includes one or more sets of instructions configured to direct a computer (which may include a plurality of computing devices, modules, or the like) to detect a blood flow signal of an individual, form a blood flow waveform based on the blood flow signal, analyze the blood flow waveform with a respiratory parameter analysis module, and determine the one or more respiratory parameters through the analyzing operation.

### BRIEF DESCRIPTION OF THE DRAWINGS

FIG. 1 illustrates a simplified block diagram of a system for determining one or more respiratory parameters, according to an embodiment of the present disclosure.

FIG. 2 illustrates a simplified block diagram of a blood flow detection device, according to an embodiment of the present disclosure.

FIG. 3 illustrates a simplified schematic diagram of a blood flow detection device positioned on an individual, according to an embodiment of the present disclosure.

FIG. 4 illustrates a blood flow waveform over time, according to an embodiment of the present disclosure.

FIG. 5 illustrates a simplified block diagram of a system for determining one or more respiratory parameters, according to an embodiment of the present disclosure.

FIG. 6 illustrates a simplified view of a display, according to an embodiment of the present disclosure.

FIG. 7(a) illustrates a top plan view of a scalogram derived from a blood flow waveform, according to an embodiment of the present disclosure.

FIG. 7(b) illustrates an isometric top view of a scalogram derived from a blood flow waveform, according to an embodiment of the present disclosure.

FIG. 7(c) illustrates an exemplary scalogram derived from a signal containing two pertinent components, according to an embodiment of the present disclosure.

FIG. 7(d) illustrates a schematic of signals associated with a ridge in FIG. 7(c), and a schematic of a further wavelet decomposition of the signals, according to an embodiment of the present disclosure.

FIG. 8 illustrates an isometric view of a blood flow detection device, according to an embodiment of the present disclosure.

FIG. 9 illustrates a simplified view of a blood flow detection device configured to be secured to a finger of an individual, according to an embodiment of the present disclosure.

FIG. 10 illustrates a simplified view of a blood flow detection device configured to be secured to or, placed on, skin of an individual, according to an embodiment of the present disclosure.

FIG. 11 illustrates a simplified view of a blood flow detection device configured to be worn around a body part of an individual, according to an embodiment of the present disclosure.

FIG. 12 illustrates a simplified view of a blood flow detection device configured to be worn on a head of an individual, according to an embodiment of the present disclosure.

FIG. 13 illustrates a simplified view of a blood flow detection device configured to be worn around a body part of an individual, according to an embodiment of the present disclosure.

FIG. 14 illustrates an isometric view of a PPG system, according to an embodiment of the present disclosure.

FIG. 15 illustrates a simplified block diagram of a PPG system, according to an embodiment of the present disclosure.

FIG. 16 illustrates a flow chart of a method of determining one or more respiratory parameters, according to an embodiment of the present disclosure.

### DETAILED DESCRIPTION

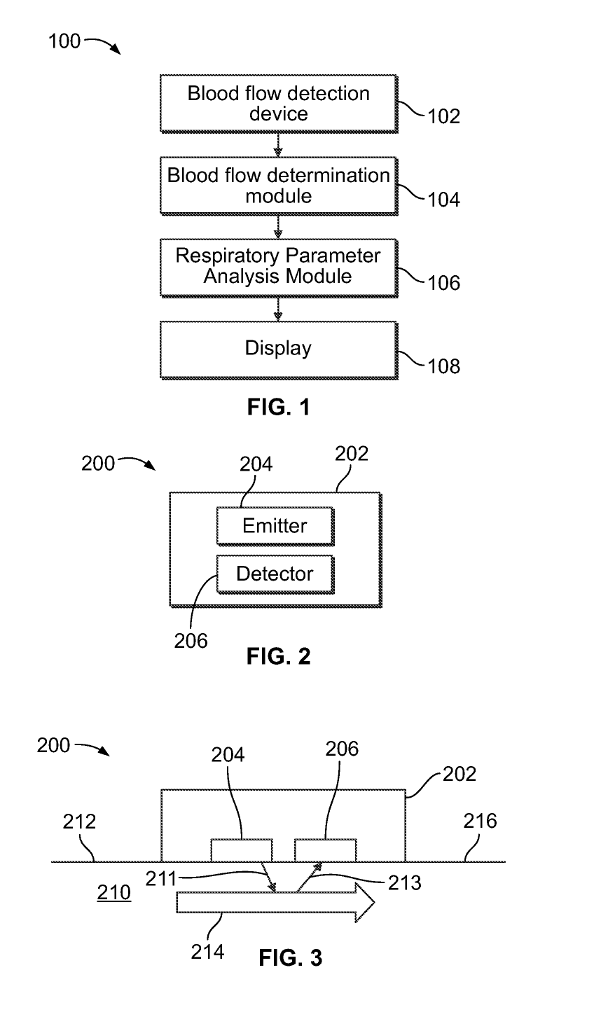

FIG. 1 illustrates a simplified block diagram of a system 100 for determining one or more respiratory parameters, according to an embodiment of the present disclosure. The respiratory parameters may include respiration rate, respiratory effort, and the like. The respiratory parameters may be derived or otherwise determined through an analysis of one or more blood flow waveforms.

The system 100 may include a blood flow detection device 102 operatively connected to and in communication with a blood flow determination module 104. The blood flow detection device 102 may be connected to the blood flow determination module 104 through a wired or cable connection. Alternatively, the blood flow detection device 102 may be wirelessly connected to the blood flow determination module 102. The blood flow detection device 102 may be a device secured to or otherwise placed on an individual that is configured to detect the blood flow of the individual. The blood flow determination module 104 is configured to receive a blood flow signal, such as a blood velocity signal, from the blood flow detection device 102. The blood flow signal may be a blood flow velocity signal or a transformation of a velocity signal including a derivative or integral. The blood flow signal may alternatively be a flow rate signal (in units of volume per time), or a mass rate signal (in units of mass per time), or any other signal indicative of fluid flow. The blood flow determination module 104 may detect and determine the blood flow signal as a modulating waveform signal, for example. As such, the blood flow determination module 104 may receive a blood flow signal from the blood flow detection device 102, and form a blood flow waveform that is based on the blood flow signal.

The blood flow determination module 104 is, in turn, operatively connected to and in communication with a respiratory parameter analysis module 106. The blood flow determination module 104 may be connected to the respiratory parameter analysis module 106 through a wired or wireless connection. The respiratory parameter analysis module 106 receives the blood flow waveform from the blood flow determination module 104 and determines one or more respiratory parameters, such as respiration rate and respiratory effort, from the blood flow waveform. For example, the respiratory parameter analysis module 106 may analyze the blood flow waveform, such as a two-dimensional blood flow waveform, and determine respiratory parameters through an analysis of the blood flow waveform, as explained below.

The respiratory parameter analysis module 106 is operatively connected to a display 108. The one or more respiratory parameters may be shown on the display 108.

The blood flow determination module 104, the respiratory parameter analysis module 106, and the display 108 may be contained within a workstation that may be or otherwise include one or more computing devices, such as standard computer hardware. The blood flow detection device 102 may be operatively connected to the workstation, such as through a cable or wireless connection. Optionally, the blood flow determination module 104 may be integrally part of the blood flow detection device 102. For example, the blood flow determination module 104 may be housed within a smart cable, adapter, or the like, that is part of a cable assembly having a sensor at one end, and a connector configured to connect to a monitor at an opposite end. Additionally, the respiratory parameter analysis module 106 may also be housed within the cable assembly. In this manner, the system 100 may be configured to connect to a device configured to display the respiratory parameters. For example, the blood flow detection device 102, the blood flow determination module 104, and the respiratory parameter analysis module 106 may be part of an assembly that connects to a device, such as a cellular or smart phone, tablet, other handheld device, laptop computer, or the like that may be configured to receive data from the assembly and show the data on a display of the device. In an embodiment, the device may be configured to download software in the form of applications configured to operate in conjunction with the assembly.

The modules 104 and 106 may include one or more control units, such as processing devices that may include one or more microprocessors, microcontrollers, integrated circuits, memory, such as read-only and/or random access memory, and the like. The modules 104 and 106 may be integrated and contained within a single housing. Alternatively, each module 104 and 106 may be contained within a respective housing, which may or may not be part of an assembly that includes the blood flow detection device 102.

The display 108 may be a cathode ray tube display, a flat panel display, such as a liquid crystal display (LCD), light-emitting diode (LED) display, a plasma display, touch screen display and interface, or any other type of monitor. The system 100 may be configured to calculate physiological parameters, such as respiratory parameters, pulse oximetry measurements, and the like, and to show information related to the physiological parameters, such as respiration rate and respiratory effort of an individual on the display 108. For example, the blood flow detection device 102 may be used in conjunction with a pulse oximetry system. In an embodiment, the blood flow detection device 102 may be integrated into a pulse oximetry sensor, and the blood flow determination module 104 and the respiratory parameter analysis module 106 may be integrated into a pulse oximetry monitor.

The system 100 may include any suitable computer-readable media used for data storage. For example, one or more of the modules 104 and 106 may include computer-readable media. The computer-readable media are configured to store information that may be interpreted by the modules 104 and 106. The information may be data or may take the form of computer-executable instructions, such as software applications, that cause a microprocessor or other such control unit within the modules 104 and 106 to perform certain functions and/or computer-implemented methods. The computer-readable media may include computer storage media and communication media. The computer storage media may include volatile and non-volatile media, removable and non-removable media implemented in any method or technology for storage of information such as computer-readable instructions, data structures, program modules or other data. The computer storage media may include, but are not limited to, RAM, ROM, EPROM, EEPROM, flash memory or other solid state memory technology, CD-ROM, DVD, or other optical storage, magnetic cassettes, magnetic tape, magnetic disk storage or other magnetic storage devices, or any other medium which may be used to store desired information and that may be accessed by components of the system.

FIG. 2 illustrates a simplified block diagram of a blood flow detection device 200, according to an embodiment of the present disclosure. The blood flow detection device 200 is an example of the blood flow detection device 102 shown in FIG. 1. The blood flow detection device 200 includes a housing 202 having an emitter 204 and a detector 206. The emitter 204 is configured to emit energy, such as light, laser, ultrasound, or the like, into patient tissue. The energy reflects or passes through blood flowing through the tissue and received by the detector 206. The energy detected by the detector may be altered as it passes through flowing blood. The blood flow determination module 104 analyzes the emitted energy and detected energy to determine blood flow characteristics, such as blood velocity.

The blood flow detection device 200 may include multiple emitters 204 and multiple detectors 206. It is to be understood that while the simplified diagram of FIG. 2 illustrates an emitter 204 and a detector 206, the illustrated emitter 204 and the detector 206 may represent multiple emitters and detectors.

FIG. 3 illustrates a simplified schematic diagram of the blood flow detection device 200 positioned on an individual 210, according to an embodiment of the present disclosure. For example, the blood flow detection device 200 may be placed on a finger, forehead, arm, or the like of an individual. The emitter 204 emits energy 211, such as a low power laser beam, into the skin 212 of the individual 210. The emitted energy 211 passes through the skin into vasculature, such as capillaries, of the individual 210 that contains flowing blood 214. The emitted energy 211 reflects or passes through the flowing blood 214. The energy 213 is then detected by the detector 206.

The blood flow detection device 200 may be a blood flow detection sensor housing an array of emitters 204 and detectors 206. Optionally, the housing 202 may support a single emitter 204 and a single detector 206. In another embodiment, the housing 202 may contain a single emitter 204 and multiple detectors 206, or multiple emitters 204 and a single detector 206. In another embodiment, the housing 202 may support only one or more detectors 206.

The blood flow detection device 200 may be configured to detect and measure the flow of blood 214 at a defined depth from a skin surface 216. Alternatively, the blood flow detection device 200 may be configured to detect and measure the average flow of blood over a region below the skin surface 216. The flowing blood 214 may be within vasculature such as a capillary bed, an artery, or vein, for example.

The blood flow detection device 200 is configured to noninvasively sense blood flow within the individual 210. The blood flow detection device 200 and the blood flow determination module 104 (shown in FIG. 1) may be included within various systems configured to non-invasively sense and determine blood flow characteristics.

As but one example, Doppler ultrasound may be used to detect and determine blood flow. Doppler ultrasound noninvasively measures blood flow by bouncing ultrasound energy off circulating blood cells. Thus, as shown in FIG. 3, the emitter 204 may be a Doppler ultrasound emitter 204 that emits the energy 211 as Doppler ultrasound energy. The detector 206 detects the reflected Doppler ultrasound energy 213 that reflects off the flowing blood 214. The blood flow determination module 104, which is in communication with the blood flow detection device 102, then determines the rate of blood flow, or blood velocity, by measuring the rate of change in the frequency or pitch of blood flow, for example. The emitter 204 and the detector 206 may be part of a transducer, for example.

The housing 202 may be a handheld instrument that is passed lightly over the skin. The transducer sends and receives ultrasound energy that may be amplified through a microphone. The ultrasound energy bounces off the blood cells. The movement of the blood cells causes a change in pitch (or frequency) of the reflected sound waves. If there is no blood flow, the pitch does not change. Information from the reflected ultrasound energy is processed by the blood flow determination module 104 to form a blood flow waveform.

Other embodiments may use angiography to detect and determine blood flow. In angiography, a radio-opaque contrast agent may be injected into the blood vessel and imaged using X-ray based techniques such as fluoroscopy. For example, the emitter 204 and detector 206 may be configured for emission and detection of X-ray energy.

In other embodiments, laser Doppler flowmetry may be used to noninvasively detect and determine blood flow. In laser Doppler flowmetry, the emitter 204 may emit laser beams as the energy 211, and the detector 206 may detect the energy 213. Laser Doppler flowmetry uses a Doppler shift in a laser beam to measure the velocity of the flowing blood 214. When the blood flow detection device 102 and the blood flow determination module 104 are configured for laser Doppler flowmetry, the emitter 204 may cross two beams of collimated, monochromatic, and coherent laser light in the flowing blood 214. The emitter 204 may form the two beams by splitting a single beam, thereby ensuring coherence between the two. Lasers with wavelengths in the visible spectrum (such as 390-750 nm) may be used. The emitter 204 may also include a transmitting optic that focuses the beams to intersect at their focal points, where they interfere and generate a set of straight fringes. As blood cells pass through the fringes, the blood cells reflect light that is then collected by receiving optics and focused on a photodetector, such as within the detector 206.

The reflected light fluctuates in intensity. The frequency of the reflected light may be equivalent to the Doppler shift between the incident and scattered light, and is thus proportional to the component of blood velocity within the path of the laser beams. If the blood flow detection device 200 is aligned with the flowing blood 214 such that the fringes are perpendicular to the flow direction, the electrical signal from the photodetector may be proportional to the full particle velocity.

Alternatively, the blood flow detection device 102 and the blood flow determination module 104 may be configured to detect and determine blood flow through various other systems and methods. For example, blood flow may be detected through optical Doppler tomography, magnetic resonance angiography, microscopic video imaging systems, laser speckle imaging, optical coherent tomography, magnetic resonance imaging, positron emission tomography, confocal microscopy, ultrasound imaging, orthogonal polarization spectral imaging, photo-acoustic methods, and the like. In each embodiment, the blood flow detection device 102, such as the blood flow detection device 200, emits energy into the flowing blood 214. The detector 206 detects the energy after it passes through or is reflected by the flowing blood 214. The blood flow determination module 104 than detects blood flow characteristics, such as blood velocity, through a comparison of the emitted energy 211 and the detected energy 213.

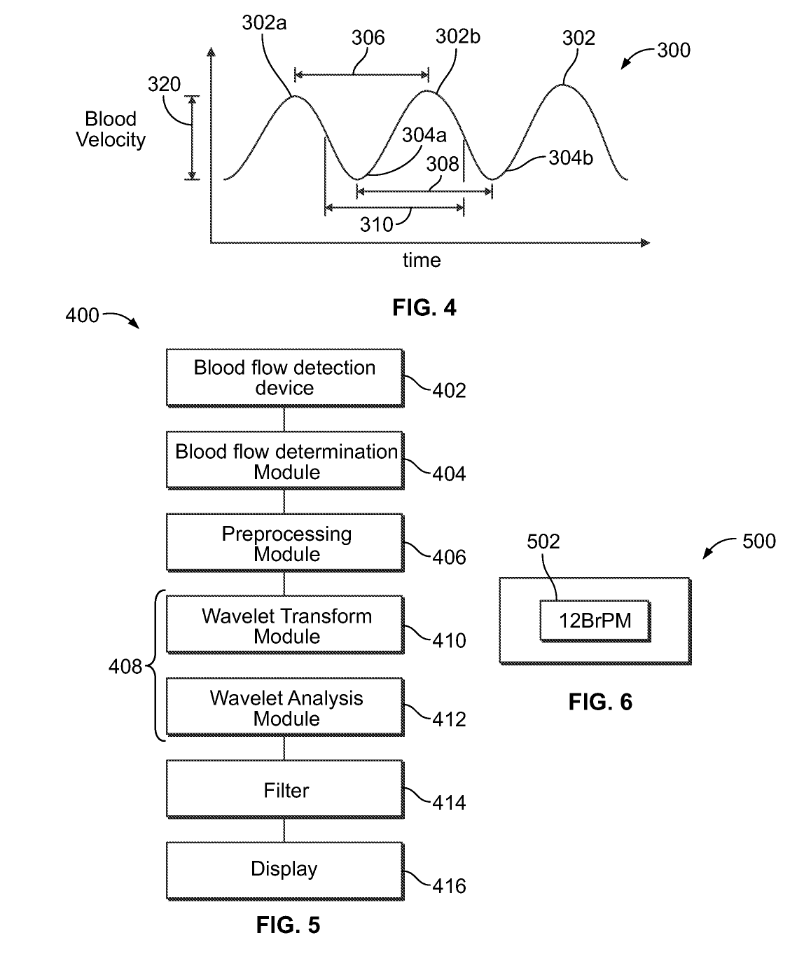

FIG. 4 illustrates a blood flow waveform 300 over time, according to an embodiment of the present disclosure. The blood flow waveform 300 may be detected by the blood flow detection device 102 and analyzed by the blood flow determination module 104 to determine the velocity of the blood flow, for example. As shown in FIG. 4, the blood flow waveform 300 oscillates over time. The blood flow waveform 300 shows areas of higher blood velocity, as shown by the peaks 302, and lower blood velocity, as shown by the troughs 304.

Referring to FIGS. 1 and 4, the respiratory parameter analysis module 106 may analyze the blood flow waveform 300 to determine respiratory parameters, such as respiration rate and respiratory effort. For example, respiration rate may be determined through the periodicity or repeating pattern of the blood flow waveform. The respiratory parameter analysis module 106 may correlate a repeating cycle of the blood flow waveform with a breath of an individual. For example, the respiratory parameter analysis module 106 may correlate a single breath with a peak span 306 between neighboring peaks 302a and 302b. Optionally, the respiratory parameter analysis module 106 may correlate a single breath with a trough span 308 between neighboring troughs 304a and 304b. Alternatively, the respiratory parameter analysis module 106 may correlate a single breath with a span 310 between other similar portions of the blood flow waveform 300. The respiratory parameter analysis module 106 may correlate the periodicity of the blood flow waveform 300 with one or more breaths of an individual to determine respiration rate. The periodicity may be between peaks, troughs, or other similar portions of the blood flow waveform 300. As such, the respiratory parameter analysis module 106 is configured to determine the respiration rate by correlating a periodicity of the blood flow waveform with breaths of an individual, or vice versa.

The respiratory parameter analysis module 106 may correlate neighboring portions of the blood flow waveform 300 (such as neighboring peaks 302a and 302b, or neighboring troughs 304a and 304b) with a single breath. Optionally, the respiratory parameter analysis module 106 may correlate a single breath with multiple neighboring portions, or vice versa.

Thus, the respiratory parameter analysis module 106 may determine a respiratory parameter, such as respiration rate, by correlating portions of a blood flow waveform with breathing characteristics of the individual. The respiratory parameter analysis module 106 may also determine other respiratory parameters by correlating other portions of the blood flow waveform with breathing characteristics of the individual.

For example, the respiratory parameter analysis module 106 may determine respiratory effort through an analysis of the blood flow waveform 300. For example, respiratory effort may be determined through an analysis of the amplitude 320 of the blood flow waveform 300. When an individual breathes harder, the amplitude 320 may increase. When an individual breathes easier, the amplitude 320 may decrease. As such, the respiratory parameter analysis module 106 may determine changes in respiratory effort by detecting changes in the amplitude 320 of the blood flow waveform 300.

Additionally, the respiratory parameter analysis module 106 may determine respiratory effort by detecting the frequency of the blood flow waveform 300. Because each peak span 306, for example, may be correlated with a single breath, the length of the peak span 306 may be indicative of respiratory effort. As the spans 306, 308, or 310 become shorter, the frequency of breathing increases. As the spans 306, 308, or 310 become longer, the frequency of breathing decreases. As such, the respiratory parameter analysis module 106 may detect the frequency of breathing through a correlation of the frequency of the blood flow waveform 300, as indicated by one or more of the spans 306, 308, or 310.

Thus, the respiratory parameter analysis module 106 may analyze the blood flow waveform 300 and determine respiratory parameters, such as respiration rate and respiratory effort, through the analysis of the blood flow waveform 300. The respiratory parameter analysis module 106 may determine respiration rate by correlating a breath with one or more of the spans 306, 308, or 310. The respiratory parameter analysis module 106 may compute breaths per minute through the correlation of the breaths within the spans 306, 308, or 310. For example, the respiratory parameter analysis module 106 may compare the span 306 with the time to determine breaths per minute. As an example, the span 306 may occur over a five second time frame. The respiratory parameter analysis module 106, through the correlation of the span 306 with a single breath, may then output a respiratory parameter of twelve breaths per minute. The respiratory parameter analysis module 106 may continually analyze the blood flow waveform 300 over time to continually update the respiratory parameter, which may be shown on the display 108 (shown in FIG. 1).

Additionally, the respiratory parameter analysis module 106 may determine the respiratory effort of the individual by analyzing the amplitude 320 and/or the frequency of the blood flow waveform 300. The respiratory effort may be directly proportional to the amplitude 320 and/or the frequency of the blood flow waveform 300. For example, as the individual breathes harder (that is, with increased effort), the amplitude 320 increases. As the individual breathes more easily (that is, with decreased effort), the amplitude 320 decreases. Similarly, as the individual breathes faster, the frequency of the blood flow signal, as represented by the spans 306, 308, or 310, increases. As the individual breathes slower, the frequency of the blood signal decreases. The respiratory parameter analysis module 106 may analyze the amplitude 320 and the frequency of the blood flow waveform 300, correlate one or both the amplitude 320 and the frequency with respiratory effort, and output the respiratory effort to be shown on the display 108. As an example, the respiratory parameter analysis module 106 may indicate changes in respiratory effort over time, such as by displaying alerts or graphics on the display 108 that the respiratory effort of the individual is increasing or decreasing over a particular time period.

Embodiments of the present disclosure provide a system and method for rapid indication of respiration rate and/or a change in the effort to breathe. Such an indication may trigger an alarm if it exceeds a threshold value. The threshold value may be an upper or lower value if, for example, a corresponding obstructive or central apnea type event occurs. In an embodiment, if respiratory effort exceeds a certain threshold, an alarm may be triggered. Similarly, if respiratory effort is below a certain threshold, an alarm may be triggered. Further, if a rate of change of respiratory effort over a predefined time exceeds a certain threshold, an alarm may be triggered.

Referring again to FIG. 1, one or more signal filters may be used to filter the blood flow signal. For example, a signal filter may be operatively connected between the blood flow detection device 102 and the blood flow determination module 104. Similarly, a signal filter may be operatively connected between the blood flow determination module 104 and the respiratory parameter analysis module 106. The signal filter(s) may filter the original blood flow signal output by the blood flow detection device 102. The signal filter(s) may include a time-domain filter, a frequency-domain filter, a wavelet transform filter, or the like.

As explained above, the respiratory parameter analysis module 106 may determine one or more respiratory parameters by correlating portions of the blood flow waveform with respiratory parameters, such as respiration rate and respiratory effort. The respiratory parameter analysis module 106 may determine respiratory parameters through various other methods, such as continuous wavelet transforms, fast Fourier transforms, temporal methods, autocorrelation, and the like.

FIG. 5 illustrates a simplified block diagram of a system 400 for determining one or more respiratory parameters, according to an embodiment of the present disclosure. The system 400 may include a blood flow detection device 402, as described above, operatively connected to and in communication with a blood flow determination module 404, such as described above. A preprocessing module 406 may be operatively connected between the blood flow determination module 404, and a respiratory parameter analysis module 408. The respiratory parameter analysis module 408 may include a wavelet transform module 410 and a wavelet analysis module 412. A filter 414 may be disposed between the respiratory parameter analysis module 408 and a display 416.

In operation, the blood flow detection device 402 detects a blood flow signal of an individual, as described above. The blood flow determination module 404 analyzes the blood flow signal to form a blood flow waveform, which may be used to determine blood flow characteristics, such as blood velocity. The blood flow determination module 404 then outputs the blood flow waveform to the respiratory parameter analysis module 408. The preprocessing module 404 may filter the blood flow waveform before it is received by the respiratory parameter analysis module 408. For example, the preprocessing module 406 may include a decimator, low pass filter, smoother, and/or the like that is configured to filter out unwanted noise or interference from the blood flow waveform.

The wavelet transform module 410 and the wavelet analysis module 412 of the respiratory parameter analysis module 408 may analyze the blood flow waveform through wavelet transform methods, as described below. The respiratory parameter analysis module 408 then outputs a respiratory parameter signal to be shown on the display 416. However, the filter 414 may first filter the respiratory parameter signal before it is shown on the display 416. The filter 414 may be a post-processing device, such as an infinite impulse response filter that receives an instantaneous respiratory parameter signal from the respiratory parameter analysis module 408 and adds and averages the instantaneous respiratory parameter signal with one or more previous signals to provide a reliable, averaged signal over time.

FIG. 6 illustrates a simplified view of a display 500, according to an embodiment of the present disclosure. The display 500 is an example of the display 108 (shown in FIG. 1) and the display 416 (shown in FIG. 5). The display 500 may be part of a monitor (such as a computer monitor, oximeter monitor, medical workstation, or the like), handheld device (such as a smart phone), or the like. The display 500 includes a display screen 502 that is configured to display one or more respiratory parameters, such as respiration rate in breaths per minute (BrPM), relative change in respiratory effort over time (for example, increasing or decreasing respiratory effort), and/or alerts related to respiration rate and/or respiratory effort. The display screen 502 may be a touch interface, such as used with various smart phones, for example.

As noted with respect to FIG. 5, the respiratory parameter analysis module 408 may include the wavelet transform module 410 and/or the wavelet analysis module 412. The blood flow determination module 404 receives a physiological blood flow signal from the blood flow detection device 402. The blood flow determination module 404 may form a blood flow waveform from the blood flow signal and determine blood flow characteristics therefrom. The respiratory parameter analysis module 408 receives the blood flow waveform and analyzes the blood flow waveform to determine respiratory parameters, such as respiration rate and/or respiratory effort. The respiratory analysis module 408 may transform the blood flow waveform into a scalogram through the wavelet transform module 410 and the wavelet analysis module 412 may determine various respiratory parameters through an analysis of the scalogram. The wavelet transform module 410 transforms the blood flow waveform received from the blood flow determination module 404 with one or more wavelet transforms to yield a scalogram, such as a rescaled blood flow scalogram.

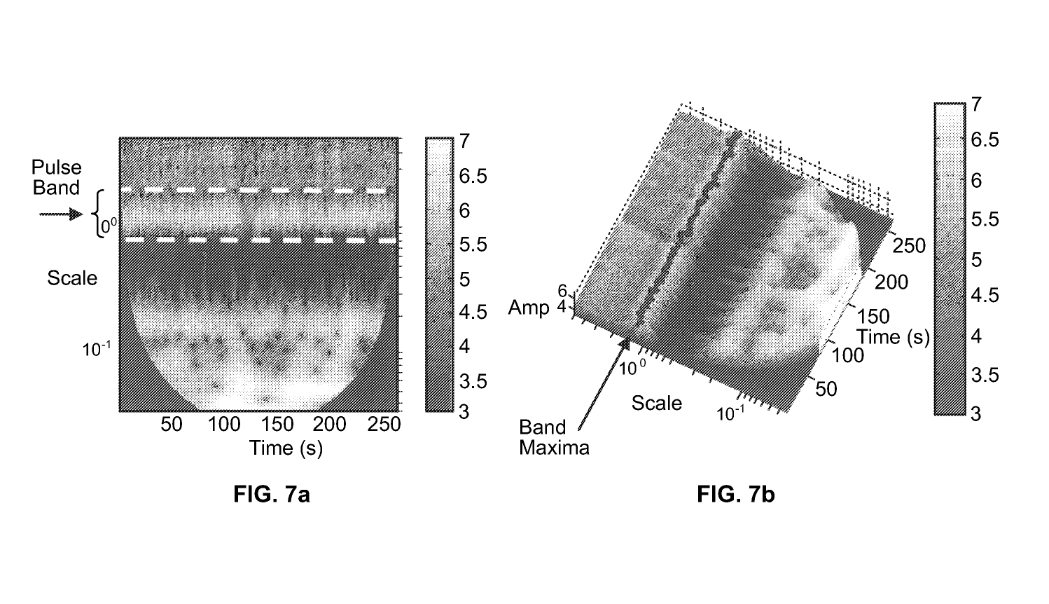

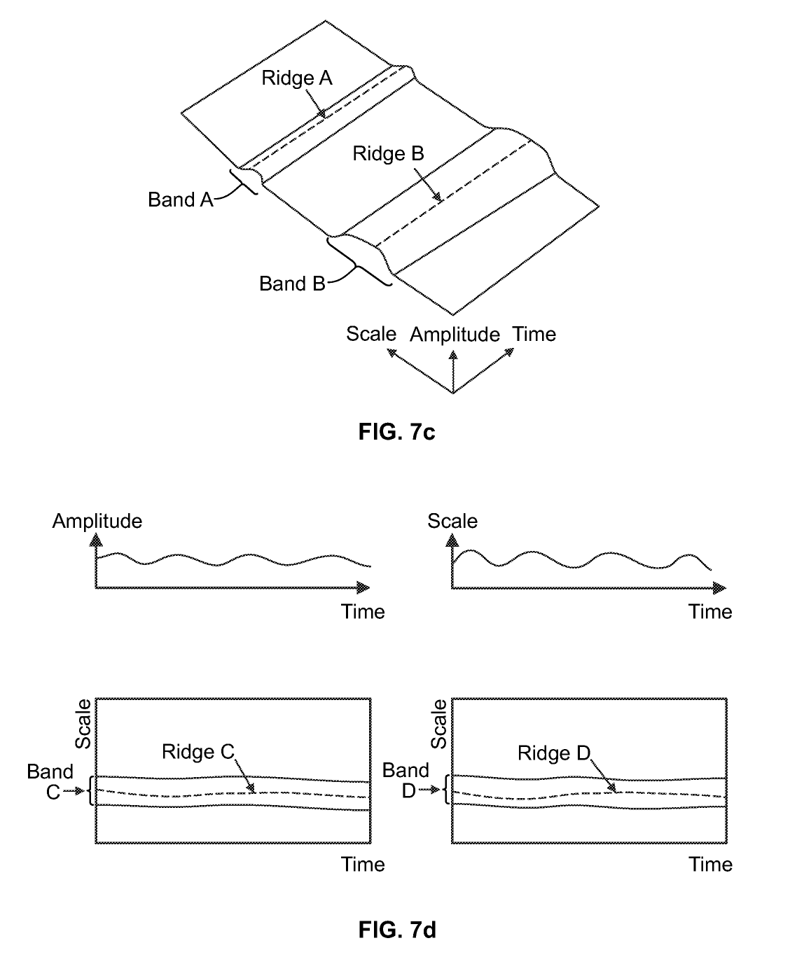

FIGS. 7(a) and 7(b) illustrate top plan and isometric views, respectively, of a scalogram derived from a blood flow waveform, according to an embodiment of the present disclosure. A blood flow waveform, such as the blood flow waveform 300 shown in FIG. 4, may be transformed using a continuous wavelet transform. Information derived from the transform of the blood flow waveform may be used to provide measurements of one or more physiological parameters.

The wavelet transform of a signal x(t) may be defined as shown in Equation (1):

$$T ( a , b ) = \frac{1}{\sqrt{a}} {\int }_{- ∞}^{+ ∞} x ( t ) {ψ}^{*} ( \frac{t - b}{a} ) d t \qquad Equation ( 1 )$$

where ψ*(t) is the complex conjugate of the wavelet function ψ(t), a is the dilation or scale parameter of the wavelet, b is the location parameter of the wavelet and x(t) is the signal under investigation. For example, x(t) may be a physiological flow signal, such as a blood flow signal or waveform, as shown in FIG. 4.

The transform given by Equation (1) may be used to construct a representation of a signal on a transform surface. The transform may be regarded as a time-scale representation. Wavelets are composed of a range of frequencies, one of which may be denoted as the characteristic frequency of the wavelet, where the characteristic frequency associated with the wavelet is inversely proportional to the scale a. One example of a characteristic frequency is the dominant frequency. Each scale of a particular wavelet may have a different characteristic frequency. The underlying mathematical detail required for the implementation within a time-scale can be found, for example, in Paul S. Addison, The Illustrated Wavelet Transform Handbook (Taylor & Francis Group 2002), which is hereby incorporated by reference herein in its entirety.

The wavelet transform decomposes a signal using wavelets, which are generally highly localized in time. A continuous wavelet transform may provide a higher resolution relative to discrete transforms, thus providing the ability to garner more information from signals that typical frequency transforms such as Fourier transforms (or any other spectral techniques) or discrete wavelet transforms. Continuous wavelet transforms allow for the use of a range of wavelets with scales spanning the scales of interest of a signal such that small scale signal components correlate well with the smaller scale wavelets and thus manifest at high energies at smaller scales in the transform. Likewise, large scale signal components correlate well with the larger scale wavelets and thus manifest at high energies at larger scales in the transform. Thus, components at different scales may be separated and extracted in the wavelet transform domain. Moreover, the use of a continuous range of wavelets in scale and time position allows for a higher resolution transform than is possible relative to discrete techniques.

In addition, transforms and operations that convert a signal or any other type of data into a spectral (i.e., frequency) domain create a series of frequency transform values in a two-dimensional coordinate system where the two dimensions may be frequency and, for example, amplitude. Wavelet transforms are further described in U.S. Pat. No. 7,944,551, entitled “Systems and Methods for a Wavelet Transform Viewer,” and U.S. Patent Application Publication No. 2010/0079279, entitled “Detecting a Signal Quality Decrease in a Measurement System,” both of which are hereby incorporated by reference in their entireties.

Referring again to Equation (1), a modulus of the transform may be defined as |T(a,b)|. As such, the energy density function of the wavelet transform (that is, the scalogram) may be rescaled as follows:

$${\vert T ( a , b ) \vert}^{*} = \frac{\vert T ( a , b ) \vert}{\sqrt{a}} \qquad Equation ( 2 )$$

The rescaled wavelet transform scalogram may be used to define ridges in wavelet space, such as when a Morlet wavelet is used. However, any suitable wavelet function may be used with embodiments of the present disclosure. The scalogram may be taken to include all suitable forms of rescaling including, but not limited to, the original unscaled wavelet representation, linear rescaling, any power of the modulus of the wavelet transform, or any other suitable rescaling. In addition, for purposes of clarity and conciseness, the term “scalogram” shall be taken to mean the wavelet transform, T(a,b) itself, or any part thereof. For example, the real part of the wavelet transform, the imaginary part of the wavelet transform, the phase of the wavelet transform, any other suitable part of the wavelet transform, or any combination thereof is intended to be conveyed by the term “scalogram”. A ridge is a locus of points of local maxima in a plane. Also, a ridge may be a path displaced from the locus of the local maxima. The rescaled wavelet transform scalogram allows for a representation in which ridges of bands on the transform surface scale directly with amplitudes of corresponding signal components. By using the rescaled transform, the direct scale relationship may take the simplified form as follows:

Aᵣ=K·Aₛ Equation (3)

where Aᵣ is the ridge amplitude, K is a constant, and Aₛ is the signal amplitude, which may be defined as the distance from peak-to-trough. For a sinusoidal signal, Equation (3) may take the form of the following:

$${A}_{r} = \frac{\sqrt[4]{π}}{2} {A}_{s} \qquad Equation ( 4 )$$

Accordingly, the ridge amplitude may be related to the signal amplitude by a constant (K) of 0.67. That is, K=\sqrt{π}/2. However, other constants may be experimentally or empirically derived, based on various factors, such as the type of wavelet transform being used.

Wavelet transform features may be extracted from the wavelet decomposition of signals. For example, wavelet decomposition of physiological blood flow waveforms may be used to provide clinically useful information.

Pertinent repeating features in a signal give rise to a time-scale band in wavelet space or a rescaled wavelet space. For example, the pulse component of a physiological pressure signal produces a dominant band in wavelet space at or around the pulse frequency. FIGS. 7(a) and 7 (b) illustrate two views of an illustrative scalogram derived from a blood flow waveform, according to an embodiment of the present disclosure. The figures show an example of the band caused by the pulse component in such a signal. The pulse band is located between the dashed lines in the plot of FIG. 7(a). The pulse band is formed from a series of dominant coalescing features across the scalogram. The pulse band is more clearly seen as a raised band across the transform surface in FIG. 7(b) located within the region of scales indicated by the arrow in the plot. The maxima of the pulse band with respect to the scale is the ridge. The locus of the ridge is shown as a black curve on top of the band in FIG. 7(b). By employing a suitable rescaling of the scalogram, such as that given in equation (2), the ridges found in wavelet space may be related to the instantaneous frequency of the signal. In this way, the pulse rate may be obtained from the signal. Instead of rescaling the scalogram, a suitable predefined relationship between the scale obtained from the ridge on the wavelet surface and the actual pulse rate may also be used to determine the pulse rate.

By mapping the time-scale coordinates of the pulse ridge onto the wavelet phase information gained through the wavelet transform, individual pulses may be captured. As such, both times between individual pulses and the timing of components within each pulse may be monitored and used to detect heart beat anomalies, measure arterial system compliance, or perform any other suitable calculations or diagnostics.

FIG. 7(c) illustrates an exemplary scalogram derived from a signal containing two pertinent components, according to an embodiment of the present disclosure. As noted above, pertinent repeating features in the signal give rise to a time-scale band in wavelet space or a rescaled wavelet space. For a periodic signal, the band remains at a constant scale in the time-scale plane. For many real signals, especially biological signals, the band may be non-stationary—varying in scale, amplitude, or both over time. As shown in FIG. 7(c), the two pertinent components lead to two bands in the transform space. The bands are labeled band A and band B on the three-dimensional schematic of the wavelet surface. In an embodiment, the band ridge is defined as the locus of the peak values of the bands with respect to scale. For purposes of clarity, band B may be referred to as the “primary band”. In addition, it may be assumed that the system from which the signal originates, and from which the transform is subsequently derived, exhibits some form of coupling between the signal components in band A and band B. When noise or other erroneous features are present in the signal with similar spectral characteristics of the features of band B, then the information within band B can become ambiguous (for example, obscured, fragmented, or missing). As such, the ridge of band A may be followed in wavelet space and extracted either as an amplitude signal or a scale signal which may be referred to as the “ridge amplitude perturbation” (RAP) signal and the “ridge scale perturbation” (RSP) signal, respectively. The RAP and RSP signals may be extracted by projecting the ridge onto the time-amplitude or time-scale planes, respectively.

FIG. 7(d) illustrates a schematic of signals associated with a ridge in FIG. 7(c), and a schematic of a further wavelet decomposition of the signals, according to an embodiment of the present disclosure. The top plots of FIG. 7(d) illustrate a schematic of the RAP and RSP signals associated with ridge A in FIG. 7(c). Below the RAP and RSP signals are schematics of a further wavelet decomposition of the newly derived signals. The secondary wavelet decomposition allows for information in the region of band B in FIG. 7(c) to be made available as band C and band D. The ridges of bands C and D may serve as instantaneous time-scale characteristic measures of the signal components causing bands C and D. This technique, which may be referred to as secondary wavelet feature decoupling (SWFD), may allow information concerning the nature of the signal components associated with the underlying physical process causing the primary band B (shown in FIG. 7(c)) to be extracted when band B itself is obscured in the presence of noise or other erroneous signal features.

In this manner, the blood flow waveform may be decoupled into component parts, such as bands A, B, C, or D. The wavelet transform module 410 of the respiratory parameter analysis module 408 (shown in FIG. 5) generates the scalogram, which decouples components of the blood flow waveform from one another. The wavelet analysis module 412 (also shown in FIG. 5) then analyzes the scalogram to determine peak-to-peak spans, trough-to-trough spans, amplitudes, frequencies, and/or the like to determine respiratory parameters, such as respiration rate, respiratory effort, and the like. For example, the wavelet analysis module 412 may detect peaks in neighboring bands associated with peak blood velocity to generate a respiration rate, in a similar manner as described above.

The respiratory parameter analysis module 408 may determine respiratory parameters by transforming the blood flow waveform into a scalogram through continuous wavelet transforms. However, embodiments of the present disclosure may determine respiratory parameters through various other methods, such as autocorrelation, fast Fourier transforms, temporal methods, and the like.

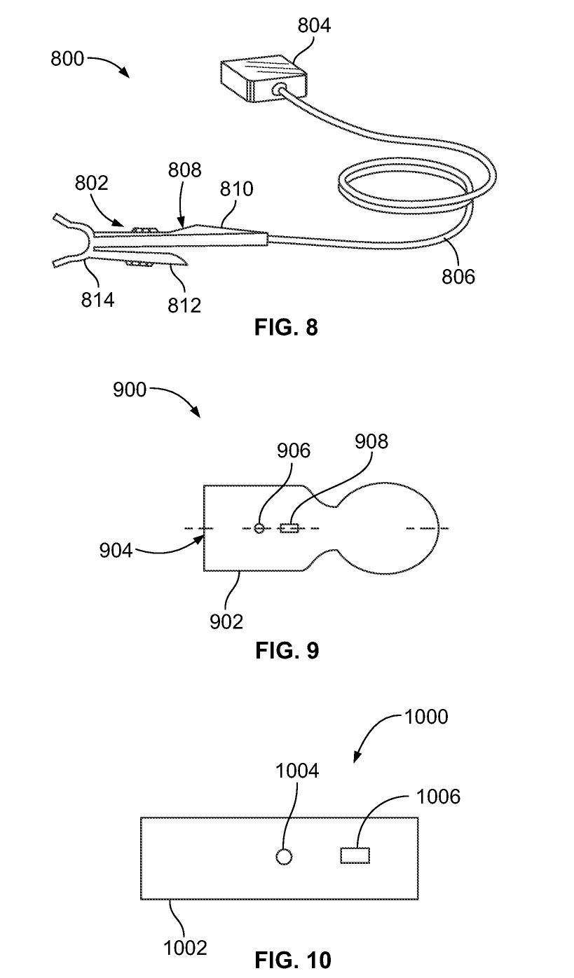

FIG. 8 illustrates an isometric view of a blood flow detection device 800, according to an embodiment of the present disclosure. The blood flow detection device 800 may be used with respect to any of the systems described above.

The blood flow detection device 800 includes a sensor 802 connected to a connector 804 through a cable assembly 806. The sensor 802 may be a clip-style sensor including a housing 808 having opposed beams 812 pivotally connected to one another through a hinge member 814. The opposed beams 812 and 814 are configured to receive an individual's finger therebetween. The housing 808 may also include one or more emitters and detectors, as described above with respect to FIG. 2.

The connector 804 is configured to removably connect the blood flow detection device 800 to a monitor, such as a workstation monitor, an oximetry monitor, a computer, a handheld device, such as a smart phone, or the like. For example, the connector 804 may be a reciprocal plug configured to mate with a connector port of a smart phone. The cable assembly 806 may include an adaptor or housing that houses a blood flow determination module, such as the blood flow determination modules 104 or 404, and/or a respiratory parameter analysis module, such as the respiratory parameter analysis module 106 or 408. Thus, the systems 100 and 400 (shown in FIGS. 1 and 4, respectively) may include components that are housed entirely within an assembly of the blood flow detection device 800. Accordingly, the blood flow detection device 800 may be connected to a device configured to display data, such as a smart phone, in which the detection and determination processing occurs in the assembly, as opposed to the device, which may otherwise not be specifically designed for respiratory parameter determination. However, it is to be understood that the assembly shown in FIG. 8 may optionally be a detection device that is configured to be connected to a separate and distinct monitor that includes a respiratory parameter analysis module, for example.

Additionally, the blood flow detection device 800 may also be configured for pulse oximetry. As such, in addition to one or more emitters and one or more emitters configured to detect and determine blood flow measurements, the sensor 802 may include one or more emitters and one or more detectors configured to detect pulse oximetry signals. The pulse oximetry or photoplethsymogram (PPG) signal may be analyzed by a monitor to determine respiration rate or respiratory effort, such as described in U.S. Pat. No. 7,035,679, entitled “Wavelet-Based Analysis of Pulse Oximetry Signals,” and U.S. Pat. No. 8,255,029, entitled “Method of Analyzing and Processing Signals,” both of which are hereby incorporated by reference in their entireties. In this manner, the PPG or pulse oximetry signal may be analyzed to determine respiration rate and/or respiratory effort, and the blood flow waveform may be analyzed to determine respiration rate and/or respiratory effort, as described above. The independent determinations of respiration rate and/or respiratory effort through analysis of the PPG or oximetry signal and the blood flow waveform may be used as an accuracy or confidence check. That is, if a determination of respiration rate and/or respiratory effort through an analysis of the PPG signal is consistent with a determination of respiration rate and/or respiratory effort through an analysis of the blood flow waveform, then a high confidence in the accuracy of the determinations exists. If the two determinations are inconsistent or otherwise differ, then a lower confidence in the accuracy exists.

FIG. 9 illustrates a simplified view of a blood flow detection device 900 configured to be secured to a finger of an individual, such as a patient within a medical facility, according to an embodiment of the present disclosure. The blood flow detection device 900 includes a housing 902 defining an internal chamber 904 configured to receive a finger of the individual. An emitter 906 and detector 908 are secured to the housing 902 and are configured to emit and detect, respective, energy, as described above with respect to FIGS. 1-4. The blood flow detection device 900 may be used as any of the blood flow detection devices shown in FIGS. 1, 2, and 5, for example.

While the blood flow detection device 900 is shown as being configured to be positioned on a finger of a patient, the blood flow detection device 900 may be sized and shaped differently, and configured to be positioned with respect to other patient anatomy, such as an arm, neck, forehead, or the like.

FIG. 10 illustrates a simplified view of a blood flow detection device 1000 configured to be secured to, or placed on, skin of a patient, according to an embodiment of the present disclosure. The blood flow detection device 1000 may include a flexible strap 1002, such as an elastomeric strap, bandage, strip, or the like, that is configured to be positioned on an anatomical structure of a patient, such as a forehead. The flexible strap 1002 supports an emitter 1004 and a detector 1006, as described above. The flexible strap 1002 is configured to be positioned on the patient anatomy and aligned with respect to underlying vasculature, such as a carotid artery, vein in the forehead, femoral artery, or the like. The blood flow detection device 1000 may be used as any of the blood flow detection devices shown in FIGS. 1, 2, and 5, for example.

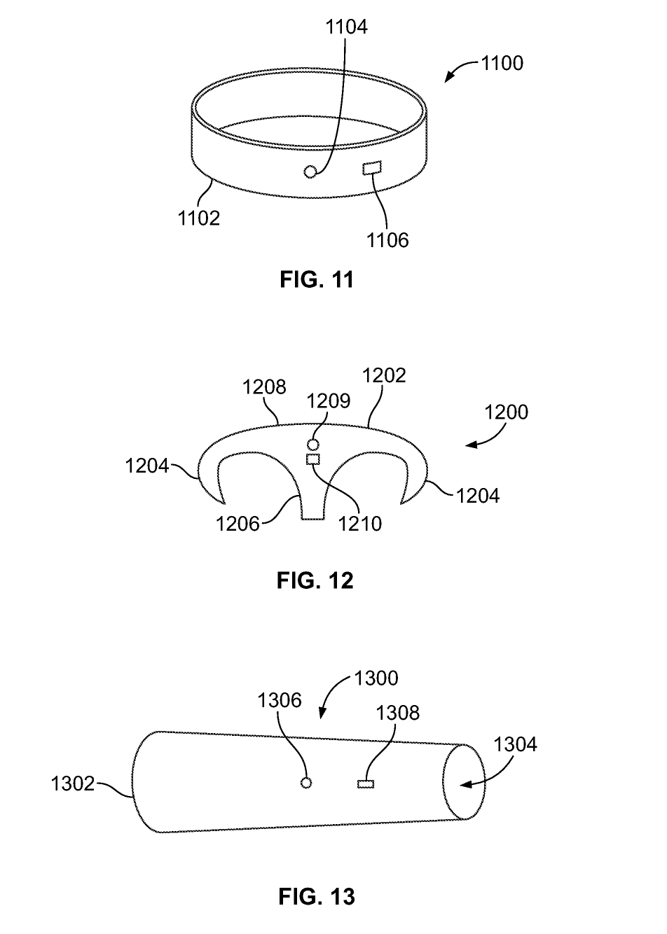

FIG. 11 illustrates a simplified view of a blood flow detection device 1100 configured to be worn around a body part of a patient, according to an embodiment of the present disclosure. The blood flow detection device 1100 may be formed of an annular flexible band 1102, such as an elastomeric headband, that is configured to be positioned on patient anatomy. The flexible band 1102 supports an emitter 1104 and a detector 1106, as described above. The flexible band 1102 is configured to be positioned on patient anatomy and aligned with respect to underlying vasculature, such as a vein or artery in the forehead. The blood flow detection device 1100 may be used as any of the blood flow detection devices shown in FIGS. 1, 2, and 5, for example.

FIG. 12 illustrates a simplified view of a blood flow detection device 1200 configured to be worn on a head of a patient, according to an embodiment of the present disclosure. The blood flow detection device 1200 may be a headset 1202 having opposed lateral supports 1204 connected to a nose support 1206 by an upper band 1208. The headset 1200 is configured to be positioned on a head of a patient, such that the nose support 1206 is positioned over a portion of a nose, and the lateral supports 1204 are positioned on sides of the patient's head. In this manner, the headset 1202 is configured to be reliably and repeatedly positioned in a similar orientation on the head time and time again. The headset 1202 is configured to be repeatedly positioned with respect to the head so that an emitter 1209 and detector 1210, as described above, are located at the same position with a high degree of accuracy. While shown on the upper band 1208, the emitter 1209 and detector 1210 may be positioned at various other locations of the blood flow detection device 1200. The blood flow detection device 1200 may be used as any of the blood flow detection devices shown in FIGS. 1, 2, and 5, for example.

FIG. 13 illustrates a simplified view of a blood flow detection device 1300 configured to be worn around a body part of a patient, according to an embodiment of the present disclosure. The blood flow detection device 1300 may be a flexible sleeve or cuff 1302 defining an internal passage 1304. The sleeve or cuff 1302 may be positioned over a forearm, shin, thigh, or the like. Similar to the other devices, the blood flow detection device 1300 includes an emitter 1306 and a detector 1308 configured to be aligned with respect to patient vasculature. The blood flow detection device 1300 may be used as any of the blood flow detection devices shown in FIGS. 1, 2, and 5, for example.

Referring to FIGS. 8-13, the blood flow detection devices may be used in conjunction with pulse oximetry sensors. For example, the blood flow detection devices may also include pulse oximetry or PPG sensors and emitters. Further, the blood flow detection devices may include more emitters and/or detectors than shown.

The blood flow detection devices may include a coupling agent (not shown) that is configured to allow the transmission of both acoustic energy and light therethrough. The coupling agent may be any type of coupling agent that is configured to allow the transmission of both acoustic energy and light therethrough, such as, but not limited to, a gel media, a cream, a fluid, a paste, an ointment, an ultrasound gel, and/or the like. In some embodiments, the blood flow detection devices may include a sponge (not shown) or other matrix device that is impregnated with the coupling agent for holding the coupling agent. Exemplary coupling agents and housings configured for use as physiological signal detection units are described in U.S. patent application Ser. No. 13/612,160, filed on Sep. 12, 2012, entitled “Photoacoustic Sensor System,” which is hereby incorporated by reference in its entirety.

The blood flow detection devices may include an adhesive configured to affix the devices to skin of an individual. The adhesive may thus further secure the devices in position with respect to patient anatomy. Any type of adhesive may be used. In some embodiments, the adhesive is specifically designed to adhere to human skin. Moreover, in addition or alternative to the adhesive, the devices may be configured to be affixed to the patient's skin using any other suitable affixing structure, such as, but not limited to, using suction, an intermediate bracket that is affixed to the patient's skin (using any suitable affixing structure) and is configured to hold the devices, and/or the like. In some alternative embodiments, no affixing structure is used besides the devices themselves. For example, the devices may include a housing having an ear clip configured to hold the devices with respect to a temporal artery, as described in U.S. patent application Ser. No. 13/618,227, filed on Sep. 14, 2012, entitled “Sensor System,” which is hereby incorporated by reference in its entirety.

As noted, embodiments of the present disclosure may be used in conjunction with a PPG or pulse oximetry system. The embodiments may be integrated with the PPG or pulse oximetry system, or separate and distinct therefrom.

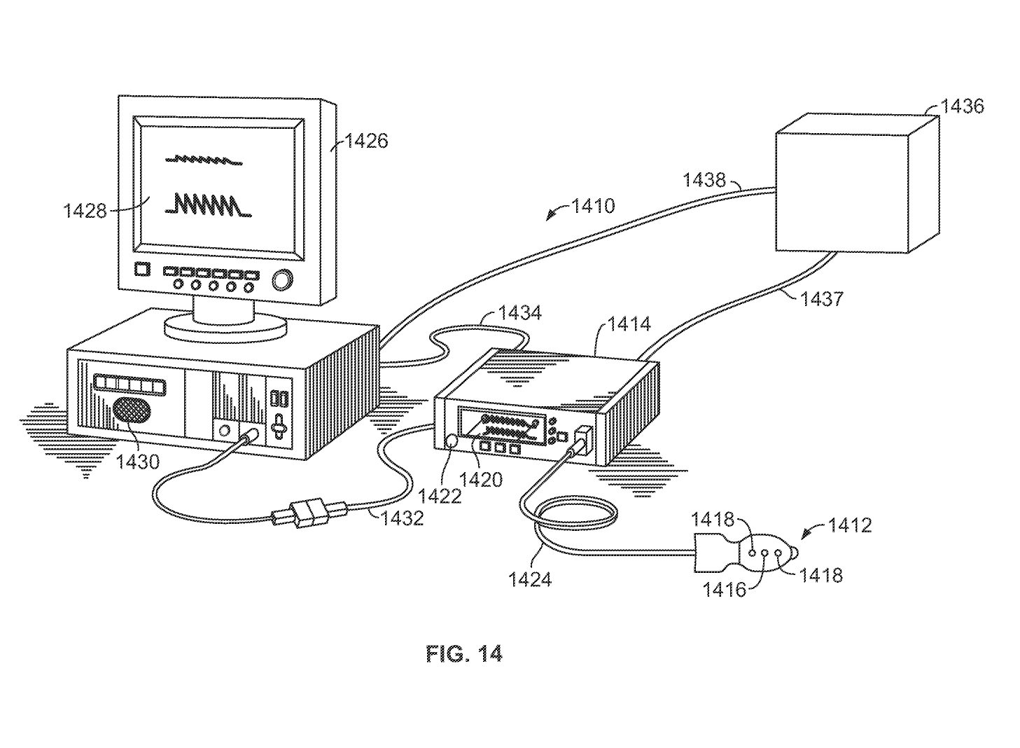

FIG. 14 illustrates an isometric view of a PPG system 1410, according to an embodiment of the present disclosure. The PPG system 1410 may be in communication with, or part of, the system 100, shown in FIG. 1 or the system 400, shown in FIG. 5. The PPG system 1410 may be a pulse oximetry system, for example. The system 1410 may include a PPG sensor 1412 and a PPG monitor 1414. The PPG sensor 1412 may include an emitter 1416 configured to emit light into tissue of a patient. For example, the emitter 1416 may be configured to emit light at two or more wavelengths into the tissue of the patient. The PPG sensor 1412 may also include spaced-apart photodetectors 1418 that are configured to detect the emitted light from the emitter 1416 that emanates from the tissue after passing through the tissue. The photodetectors 1418 may be equidistant, but on opposite sides, from the emitter 1116.

The system 1410 may include a plurality of sensors forming a sensor array in place of the PPG sensor 1412. Each of the sensors of the sensor array may be a complementary metal oxide semiconductor (CMOS) sensor, for example. Alternatively, each sensor of the array may be a charged coupled device (CCD) sensor. In another embodiment, the sensor array may include a combination of CMOS and CCD sensors. The CCD sensor may include a photoactive region and a transmission region configured to receive and transmit, while the CMOS sensor may include an integrated circuit having an array of pixel sensors. Each pixel may include a photodetector and an active amplifier.

The emitter 1416 and the photodetectors 1418 may be configured to be located on opposite sides of a digit, such as a finger or toe, in which case the light that emanates from the tissue passes completely through the digit. The emitter 1416 and the photodetectors 1418 may be arranged so that light from the emitter 1416 penetrates the tissue and is reflected by the tissue into the detector 1418, such as a sensor designed to obtain pulse oximetry data.

The sensor 1412 or sensor array may be operatively connected to and draw power from the monitor 1414, for example. Optionally, the sensor 1412 may be wirelessly connected to the monitor 1414 and include a battery or similar power supply (not shown). The monitor 1414 may be configured to calculate physiological parameters based at least in part on data received from the sensor 1412 relating to light emission and detection. Alternatively, the calculations may be performed by and within the sensor 1412 and the result of the oximetry reading may be passed to the monitor 1414. Additionally, the monitor 1414 may include a display 1420 configured to display the physiological parameters or other information about the system 1410. The monitor 1414 may also include a speaker 1422 configured to provide an audible sound that may be used in various other embodiments, such as for example, sounding an audible alarm in the event that physiological parameters are outside a predefined normal range.

The sensor 1412, or the sensor array, may be communicatively coupled to the monitor 1414 via a cable 1424. Alternatively, a wireless transmission device (not shown) or the like may be used instead of, or in addition to, the cable 1424.

The system 1410 may also include a multi-parameter workstation 1426 operatively connected to the monitor 1414. The workstation 1426 may be or include a computing sub-system 1430, such as standard computer hardware. The computing sub-system 1430 may include one or more modules and control units, such as processing devices that may include one or more microprocessors, microcontrollers, integrated circuits, memory, such as read-only and/or random access memory, and the like. The workstation 1426 may include a display 1428, such as a cathode ray tube display, a flat panel display, such as a liquid crystal display (LCD), a light-emitting diode (LED) display, a plasma display, or any other type of monitor. The computing sub-system 1430 of the workstation 1426 may be configured to calculate physiological parameters and to show information from the monitor 1414 and from other medical monitoring devices or systems (not shown) on the display 1428. For example, the workstation 1426 may be configured to display an estimate of a patient's blood oxygen saturation generated by the monitor 1414 (referred to as an SpO₂ measurement), pulse rate information from the monitor 1414, and respiration rate and/or respiratory effort determined by a respiratory parameter analysis module within the monitor 1414 or workstation 1426 on the display 1428.

The monitor 1414 may be communicatively coupled to the workstation 1426 via a cable 1432 and/or 1434 that is coupled to a sensor input port or a digital communications port, respectively and/or may communicate wirelessly with the workstation 1426. Additionally, the monitor 1414 and/or workstation 1426 may be coupled to a network to enable the sharing of information with servers or other workstations. The monitor 1414 may be powered by a battery or by a conventional power source such as a wall outlet.

The system 1410 may also include a fluid delivery device 1436 that is configured to deliver fluid to a patient. The fluid delivery device 1436 may be an intravenous line, an infusion pump, any other suitable fluid delivery device, or any combination thereof that is configured to deliver fluid to a patient. The fluid delivered to a patient may be saline, plasma, blood, water, any other fluid suitable for delivery to a patient, or any combination thereof. The fluid delivery device 1436 may be configured to adjust the quantity or concentration of fluid delivered to a patient.

The fluid delivery device 1436 may be communicatively coupled to the monitor 1414 via a cable 1437 that is coupled to a digital communications port or may communicate wirelessly with the workstation 1426. Alternatively, or additionally, the fluid delivery device 1436 may be communicatively coupled to the workstation 1426 via a cable 1438 that is coupled to a digital communications port or may communicate wirelessly with the workstation 1426.

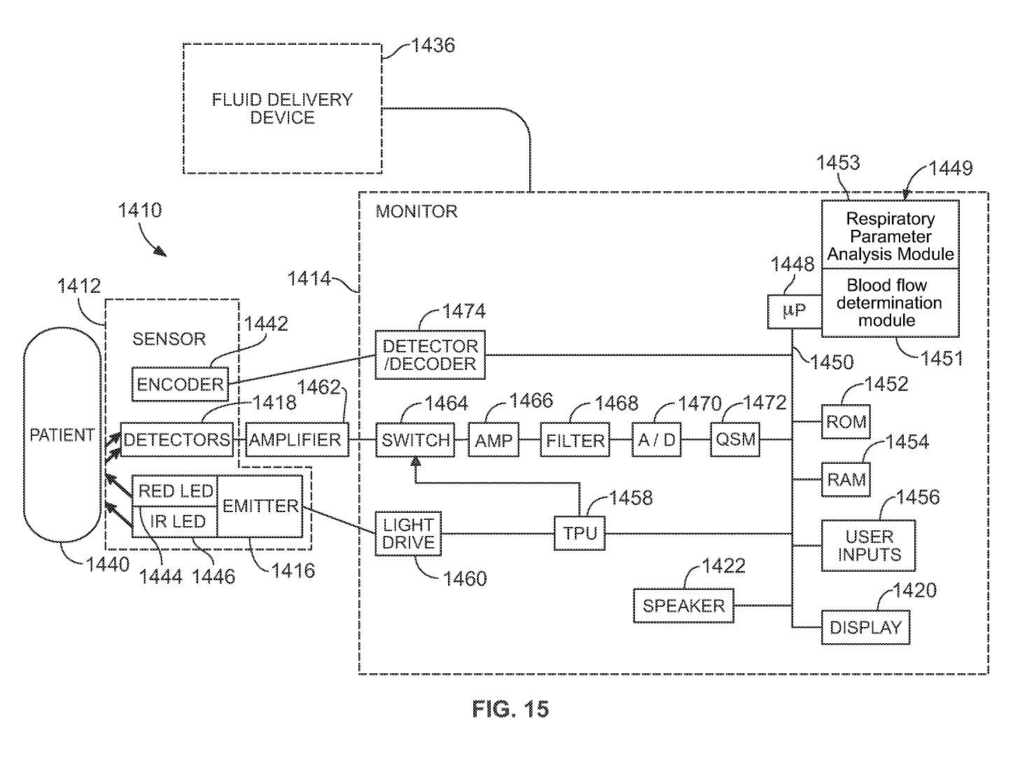

FIG. 15 illustrates a simplified block diagram of the PPG system 1410, according to an embodiment of the present disclosure. When the PPG system 1410 is a pulse oximetry system, the emitter 1416 may be configured to emit at least two wavelengths of light (for example, red and infrared) into tissue 1440 of a patient. Accordingly, the emitter 1416 may include a red light-emitting light source such as a red light-emitting diode (LED) 1444 and an infrared light-emitting light source such as an infrared LED 1446 for emitting light into the tissue 1440 at the wavelengths used to calculate the patient's physiological parameters. For example, the red wavelength may be between about 600 nm and about 700 nm, and the infrared wavelength may be between about 800 nm and about 1000 nm. In embodiments where a sensor array is used in place of single sensor, each sensor may be configured to emit a single wavelength. For example, a first sensor may emit a red light while a second sensor may emit an infrared light. Additionally, the PPG system 1410 may include emitters and detectors that are configured for detection of blood flow signals, as described above.

As discussed above, the PPG system 1410 is described in terms of a pulse oximetry system. However, the PPG system 1410 may be various other types of systems. For example, the PPG system 1410 may be configured to emit more or less than two wavelengths of light into the tissue 1440 of the patient. Further, the PPG system 1410 may be configured to emit wavelengths of light other than red and infrared into the tissue 1440. As used herein, the term “light” may refer to energy produced by radiative sources and may include one or more of ultrasound, radio, microwave, millimeter wave, infrared, visible, ultraviolet, gamma ray or X-ray electromagnetic radiation. The light may also include any wavelength within the radio, microwave, infrared, visible, ultraviolet, or X-ray spectra, and that any suitable wavelength of electromagnetic radiation may be used with the system 1410. The photodetectors 1418 may be configured to be specifically sensitive to the chosen targeted energy spectrum of the emitter 1416.

The photodetectors 1418 may be configured to detect the intensity of light at the red and infrared wavelengths. Alternatively, each sensor in the array may be configured to detect an intensity of a single wavelength. In operation, light may enter the photodetectors 1418 after passing through the tissue 1440. The photodetectors 1418 may convert the intensity of the received light into electrical signals. The light intensity may be directly related to the absorbance and/or reflectance of light in the tissue 1440. For example, when more light at a certain wavelength is absorbed or reflected, less light of that wavelength is received from the tissue by the photodetectors 1418. After converting the received light to an electrical signal, the photodetectors 1418 may send the signal to the monitor 1414, which calculates physiological parameters based on the absorption of the red and infrared wavelengths in the tissue 1440.

In an embodiment, an encoder 1442 may store information about the sensor 1412, such as sensor type (for example, whether the sensor is intended for placement on a forehead or digit) and the wavelengths of light emitted by the emitter 1416. The stored information may be used by the monitor 1414 to select appropriate algorithms, lookup tables and/or calibration coefficients stored in the monitor 1414 for calculating physiological parameters of a patient. The encoder 1442 may store or otherwise contain information specific to a patient, such as, for example, the patient's age, weight, diagnosis, and/or the like. The information may allow the monitor 1414 to determine, for example, patient-specific threshold ranges related to the patient's physiological parameter measurements, and to enable or disable additional physiological parameter algorithms. The encoder 1442 may, for instance, be a coded resistor that stores values corresponding to the type of sensor 1412 or the types of each sensor in the sensor array, the wavelengths of light emitted by emitter 1416 on each sensor of the sensor array, and/or the patient's characteristics. Optionally, the encoder 1442 may include a memory in which one or more of the following may be stored for communication to the monitor 1414: the type of the sensor 1412, the wavelengths of light emitted by emitter 1416, the particular wavelength each sensor in the sensor array is monitoring, a signal threshold for each sensor in the sensor array, any other suitable information, or any combination thereof.

Signals from the photodetectors 1418 and the encoder 1442 may be transmitted to the monitor 1414. The monitor 1414 may include a general-purpose control unit, such as a microprocessor 1448 connected to an internal bus 1450. The microprocessor 1448 may be configured to execute software, which may include an operating system and one or more applications, as part of performing the functions described herein. A read-only memory (ROM) 1452, a random access memory (RAM) 1454, user inputs 1456, the display 1420, and the speaker 1422 may also be operatively connected to the bus 1450.

The microprocessor 1448 may be operatively connected to, or include, a system 1449 that includes a blood flow determination module 1451 and a respiratory parameter analysis module 1453, such as any of those described above. The system 1449 may be integrated into the PPG system 1410. In such an embodiment, the sensor 1412 also includes one or more emitters and one or more detectors configured for use in a blood flow detection device.

The RAM 1454 and the ROM 1452 are illustrated by way of example, and not limitation. Any suitable computer-readable media may be used in the system for data storage. Computer-readable media are configured to store information that may be interpreted by the microprocessor 1448. The information may be data or may take the form of computer-executable instructions, such as software applications, that cause the microprocessor to perform certain functions and/or computer-implemented methods. The computer-readable media may include computer storage media and communication media. The computer storage media may include volatile and non-volatile media, removable and non-removable media implemented in any method or technology for storage of information such as computer-readable instructions, data structures, program modules or other data. The computer storage media may include, but are not limited to, RAM, ROM, EPROM, EEPROM, flash memory or other solid state memory technology, CD-ROM, DVD, or other optical storage, magnetic cassettes, magnetic tape, magnetic disk storage or other magnetic storage devices, or any other medium which may be used to store desired information and that may be accessed by components of the system.

The monitor 1414 may also include a time processing unit (TPU) 1458 configured to provide timing control signals to a light drive circuitry 1460, which may control when the emitter 1416 is illuminated and multiplexed timing for the red LED 1444 and the infrared LED 1446. The TPU 1458 may also control the gating-in of signals from the photodetectors 1418 through an amplifier 1462 and a switching circuit 1464. The signals are sampled at the proper time, depending upon which light source is illuminated. The received signals from the photodetectors 1418 may be passed through an amplifier 1466, a low pass filter 1468, and an analog-to-digital converter 1470. The digital data may then be stored in a queued serial module (QSM) 1472 (or buffer) for later downloading to RAM 1454 as QSM 1472 fills up. In an embodiment, there may be multiple separate parallel paths having amplifier 1466, filter 1468, and A/D converter 1470 for multiple light wavelengths or spectra received.

The microprocessor 1448 may be configured to determine the patient's physiological parameters, such as SpO₂ and pulse rate using various algorithms and/or look-up tables based on the value(s) of the received signals and/or data corresponding to the light received by the photodetectors 1418. The signals corresponding to information about a patient, and regarding the intensity of light emanating from the tissue 1440 over time, may be transmitted from the encoder 1442 to a decoder 1474. The transmitted signals may include, for example, encoded information relating to patient characteristics. The decoder 1474 may translate the signals to enable the microprocessor 1448 to determine the thresholds based on algorithms or look-up tables stored in the ROM 1452. The user inputs 1456 may be used to enter information about the patient, such as age, weight, height, diagnosis, medications, treatments, and so forth. The display 1420 may show a list of values that may generally apply to the patient, such as, for example, age ranges or medication families, which the user may select using the user inputs 1456.

The fluid delivery device 1436 may be communicatively coupled to the monitor 1414. The microprocessor 1448 may determine the patient's physiological parameters, such as a change or level of fluid responsiveness, and display the parameters on the display 1420. In an embodiment, the parameters determined by the microprocessor 1448 or otherwise by the monitor 1414 may be used to adjust the fluid delivered to the patient via fluid delivery device 1436.

As noted, the PPG system 1410 may be a pulse oximetry system. A pulse oximeter is a medical device that may determine oxygen saturation of blood. The pulse oximeter may indirectly measure the oxygen saturation of a patient's blood (as opposed to measuring oxygen saturation directly by analyzing a blood sample taken from the patient) and changes in blood volume in the skin. Ancillary to the blood oxygen saturation measurement, pulse oximeters may also be used to measure the pulse rate of a patient. Pulse oximeters measure and display various blood flow characteristics including, but not limited to, the oxygen saturation of hemoglobin in arterial blood.

A pulse oximeter may include a light sensor, similar to the sensor 1412, that is placed at a site on a patient, typically a fingertip, toe, forehead or earlobe, or in the case of a neonate, across a foot. The pulse oximeter may pass light using a light source through blood perfused tissue and photoelectrically sense the absorption of light in the tissue. For example, the pulse oximeter may measure the intensity of light that is received at the light sensor as a function of time. A signal representing light intensity versus time or a mathematical manipulation of this signal (for example, a scaled version thereof, a log taken thereof, a scaled version of a log taken thereof, and/or the like) may be referred to as the PPG signal. In addition, the term “PPG signal,” as used herein, may also refer to an absorption signal (for example, representing the amount of light absorbed by the tissue) or any suitable mathematical manipulation thereof. The light intensity or the amount of light absorbed may then be used to calculate the amount of the blood constituent (for example, oxyhemoglobin) being measured as well as the pulse rate and when each individual pulse occurs.

The light passed through the tissue is selected to be of one or more wavelengths that are absorbed by the blood in an amount representative of the amount of the blood constituent present in the blood. The amount of light passed through the tissue varies in accordance with the changing amount of blood constituent in the tissue and the related light absorption. Red and infrared wavelengths may be used because it has been observed that highly oxygenated blood will absorb relatively less red light and more infrared light than blood with lower oxygen saturation. By comparing the intensities of two wavelengths at different points in the pulse cycle, it is possible to estimate the blood oxygen saturation of hemoglobin in arterial blood.

The PPG system 1410 and pulse oximetry may be further described in United States Patent Application Publication No. 2012/0053433, entitled “System and Method to Determine SpO₂ Variability and Additional Physiological Parameters to Detect Patient Status,” United States Patent Application Publication No. 2010/0324827, entitled “Fluid Responsiveness Measure,” and United States Patent Application Publication No. 2009/0326353, entitled “Processing and Detecting Baseline Changes in Signals,” all of which are hereby incorporated by reference in their entireties.

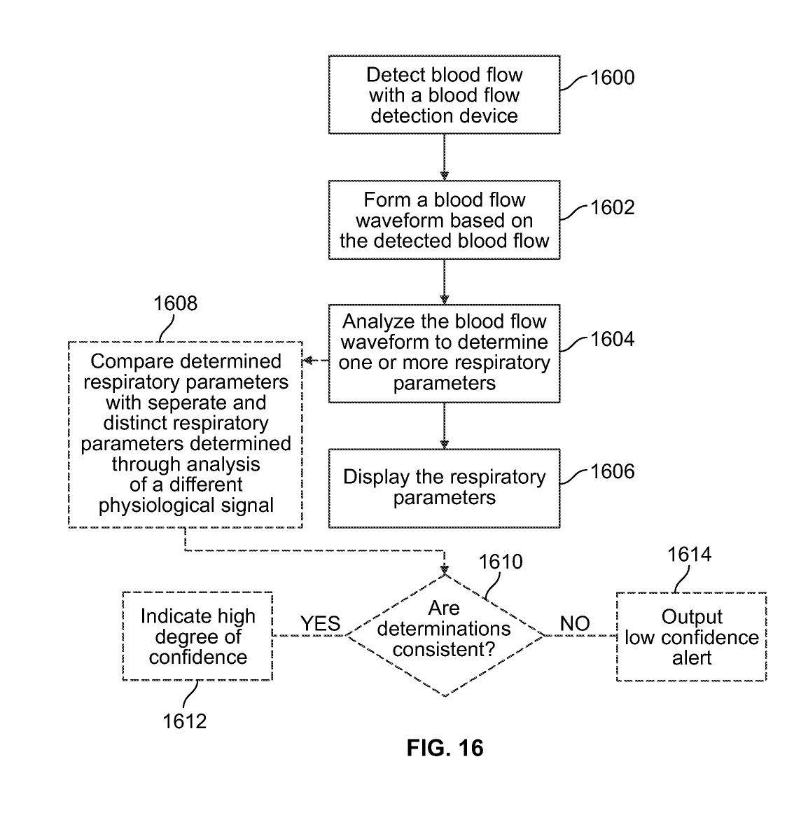

FIG. 16 illustrates a flow chart of a method of determining one or more respiratory parameters, according to an embodiment of the present disclosure. At 1600, blood flow is detected with a blood flow detection device. The blood flow signal is received by a blood flow determination module. At 1602, the blood flow determination module forms a blood flow waveform based on the detected blood flow. Then, at 1604, a respiratory parameter analysis module analyzes the blood flow waveform to determine or more respiratory parameters, such as respiration rate and/or respiratory effort. The respiratory parameter(s) are then displayed at 1606.

The method may also include additional alternative operations. For example, at 1608, after the one or more respiratory parameters have been determined at 1604, the determined respiratory parameters are compared with separate and distinct respiratory parameters that have been determined through analysis of a different physiological signal, such as a PPG signal. At 1610, a computing device or module determines whether the sets of respiratory parameters are consistent. If they are consistent, the computing device or module indicates a high degree of confidence in the accuracy of the respiratory parameters on a display at 1612. If the sets of respiratory parameters are not consistent, then at 1614, the computing device or module may output a low confidence alert on the display, speakers, and/or the like.

Various embodiments described herein provide a tangible and non-transitory (for example, not an electric signal) machine-readable medium or media having instructions recorded thereon for a processor or computer to operate a system to perform one or more embodiments of methods described herein. The medium or media may be any type of CD-ROM, DVD, floppy disk, hard disk, optical disk, flash RAM drive, or other type of computer-readable medium or a combination thereof.

The various embodiments and/or components, for example, the control units, modules, or components and controllers therein, also may be implemented as part of one or more computers or processors. The computer or processor may include a computing device, an input device, a display unit and an interface, for example, for accessing the Internet. The computer or processor may include a microprocessor. The microprocessor may be connected to a communication bus. The computer or processor may also include a memory. The memory may include Random Access Memory (RAM) and Read Only Memory (ROM). The computer or processor may also include a storage device, which may be a hard disk drive or a removable storage drive such as a floppy disk drive, optical disk drive, and the like. The storage device may also be other similar means for loading computer programs or other instructions into the computer or processor.

As used herein, the term “computer” or “module” may include any processor-based or microprocessor-based system including systems using microcontrollers, reduced instruction set computers (RISC), application specific integrated circuits (ASICs), logic circuits, and any other circuit or processor capable of executing the functions described herein. The above examples are exemplary only, and are thus not intended to limit in any way the definition and/or meaning of the term “computer” or “module.”

The computer or processor executes a set of instructions that are stored in one or more storage elements, in order to process input data. The storage elements may also store data or other information as desired or needed. The storage element may be in the form of an information source or a physical memory element within a processing machine.

The set of instructions may include various commands that instruct the computer or processor as a processing machine to perform specific operations such as the methods and processes of the various embodiments of the subject matter described herein. The set of instructions may be in the form of a software program. The software may be in various forms such as system software or application software. Further, the software may be in the form of a collection of separate programs or modules, a program module within a larger program or a portion of a program module. The software also may include modular programming in the form of object-oriented programming. The processing of input data by the processing machine may be in response to user commands, or in response to results of previous processing, or in response to a request made by another processing machine.

As used herein, the terms “software” and “firmware” are interchangeable, and include any computer program stored in memory for execution by a computer, including RAM memory, ROM memory, EPROM memory, EEPROM memory, and non-volatile RAM (NVRAM) memory. The above memory types are exemplary only, and are thus not limiting as to the types of memory usable for storage of a computer program.

As described above, embodiments of the present disclosure provides systems and methods for determining respiratory parameters from blood flow signals. The systems and methods may be efficiently integrated into existing systems, such as pulse oximetry systems. Further, the systems and methods do not interfere with the operation of the existing systems and methods.

While various spatial and directional terms, such as top, bottom, lower, mid, lateral, horizontal, vertical, front, and the like may be used to describe embodiments, it is understood that such terms are merely used with respect to the orientations shown in the drawings. The orientations may be inverted, rotated, or otherwise changed, such that an upper portion is a lower portion, and vice versa, horizontal becomes vertical, and the like.

It is to be understood that the above description is intended to be illustrative, and not restrictive. For example, the above-described embodiments (and/or aspects thereof) may be used in combination with each other. In addition, many modifications may be made to adapt a particular situation or material to the teachings without departing from its scope. While the dimensions, types of materials, and the like described herein are intended to define the parameters of the disclosure, they are by no means limiting and are exemplary embodiments. Many other embodiments will be apparent to those of skill in the art upon reviewing the above description. The scope of the disclosure should, therefore, be determined with reference to the appended claims, along with the full scope of equivalents to which such claims are entitled. In the appended claims, the terms “including” and “in which” are used as the plain-English equivalents of the respective terms “comprising” and “wherein.” Moreover, in the following claims, the terms “first,” “second,” and “third,” etc. are used merely as labels, and are not intended to impose numerical requirements on their objects. Further, the limitations of the following claims are not written in means—plus-function format and are not intended to be interpreted based on 35 U.S.C. §112, sixth paragraph, unless and until such claim limitations expressly use the phrase “means for” followed by a statement of function void of further structure.

What is claimed is:

1. A system for determining one or more respiratory parameters of an individual, the system comprising:
a blood flow detection device configured to detect a blood flow signal of the individual;
a blood flow determination module configured to form a blood flow waveform based on the blood flow signal; and
a respiratory parameter analysis module configured to analyze the blood flow waveform and determine the one or more respiratory parameters from an analysis of the blood flow waveform.

2. The system of claim 1, wherein the one or more respiratory parameters includes respiration rate.

3. The system of claim 2, wherein the respiratory parameter analysis module is configured to determine the respiration rate by correlating a periodicity of the blood flow waveform with breaths of the individual.

4. The system of claim 1, wherein the one or more respiratory parameters includes respiratory effort.

5. The system of claim 4, wherein the respiratory parameter analysis module is configured to determine the respiratory effort through a determination of one or both of an amplitude or frequency of the blood flow waveform.

6. The system of claim 5, wherein the respiratory effort is directly proportional to one or both the amplitude or the frequency of the blood flow waveform.

7. The system of claim 1, wherein the respiratory parameter analysis module is configured to determine the one or more respiratory parameters through a correlation of portions of the blood flow waveform with breathing characteristics of the individual.

8. The system of claim 1, wherein the respiratory parameter analysis module includes:
a wavelet transform module configured to transform the blood flow waveform into a scalogram using at least one wavelet transform; and
a wavelet analysis module configured to determine the one or more respiratory parameters through an analysis of the scalogram.

9. The system of claim 1, wherein the blood flow detection device is configured to detect the blood flow signal through one or more of Doppler ultrasound, angiography, laser Doppler flowmetry, optical Doppler tomography, magnetic resonance angiography, microscopic video imaging, laser speckle imaging, optical coherent tomography, positron emission tomography, confocal microscopy, ultrasound imaging, or orthogonal polarization spectral imaging.

10. A method for determining one or more respiratory parameters of an individual, the method comprising:
detecting a blood flow signal of an individual with a blood flow detection device;
forming a blood flow waveform based on the blood flow signal with a blood flow determination module;
analyzing the blood flow waveform with a respiratory parameter analysis module; and
determining, with the respiratory analysis module, the one or more respiratory parameters through the analyzing operation.

11. The method of claim 10, wherein the one or more respiratory parameters includes respiration rate.

12. The method of claim 11, wherein the determining operation comprises determining the respiration rate by correlating a periodicity of the blood flow waveform with breaths of the individual.

13. The method of claim 10, wherein the one or more respiratory parameters includes respiratory effort.

14. The method of claim 13, wherein the determining operation comprises determining one or both of an amplitude or frequency of the blood flow waveform.

15. The method of claim 10, wherein the analyzing operation comprises transforming the blood flow waveform into a scalogram using at least one wavelet transform, and wherein the determining operation comprises determining the one or more respiratory parameters through an analysis of the scalogram.

16. The method of claim 1, wherein the detecting operation comprises using one or more of Doppler ultrasound, angiography, laser Doppler flowmetry, optical Doppler tomography, magnetic resonance angiography, microscopic video imaging, laser speckle imaging, optical coherent tomography, positron emission tomography, confocal microscopy, ultrasound imaging, or orthogonal polarization spectral imaging.

17. A tangible and non-transitory computer readable medium that includes one or more sets of instructions configured to direct a computer to:
detect a blood flow signal of an individual;
form a blood flow waveform based on the blood flow signal;
analyze the blood flow waveform with a respiratory parameter analysis module; and
determine the one or more respiratory parameters through the analyzing operation.

18. The tangible and non-transitory computer readable medium of claim 17, wherein the one or more instructions are further configured to direct the computer to determine a respiration rate by correlating a periodicity of the blood flow waveform with breaths of the individual.

19. The tangible and non-transitory computer readable medium of claim 17, wherein the one or more instructions are further configured to direct the computer to determine one or both of an amplitude or frequency of the blood flow waveform to determine a respiratory effort.

20. The tangible and non-transitory computer readable medium of claim 17, wherein the one or more instructions are further configured to direct the computer to transform the blood flow waveform into a scalogram using at least one wavelet transform, and determine the one or more respiratory parameters through an analysis of the scalogram.
